(19) United States
(12) Patent Application Publication
(10) Pub. No.: US 2015/0024453 A1
(43) Pub. Date: Jan. 22, 2015

(54) INTEGRATED PROCESS FOR DUAL BIOCATALYTIC CONVERSION OF CO2 GAS INTO BIO-PRODUCTS BY ENZYME ENHANCED HYDRATION AND BIOLOGICAL CULTURE

(71) Applicant: CO2 SOLUTIONS INC., Québec (CA)

(72) Inventors: Sylvie Fradette, Pintendre (CA); Chantal Guimond, Quebec (CA); Eric Madore, Quebec (CA); Glenn R. Kelly, Trois-Rivieres (CA); Jonathan A. Carley, Etobicoke (CA); Geert F. Versteeg, Enschede (NL)

(21) Appl. No.: 14/372,771

(22) PCT Filed: Jan. 17, 2013
(86) PCT No.: PCT/CA2013/050029

§ 371 Date: Jul. 17, 2014

Related U.S. Application Data
(60) Provisional application No. 61/587,341, filed on Jan. 17, 2012.

(51) Int. Cl. C12P 3/00; C12N 9/88

(52) U.S. Cl. CPC C12P 3/00; C12N 9/88; C12Y 402/01001
USPC 435/168; 435/294.1; 435/232

(57) ABSTRACT

A method, process, apparatus, use and formulation for dual biocatalytic conversion of CO₂ containing gas into carbon containing bio-products by enzymatic hydration of CO₂ into bicarbonate ions in the presence of carbonic anhydrase and metabolic conversion of the bicarbonate ions into carbon containing bio-products in a biological culture. The dual biocatalytic conversion may be relatively constant with controlling a feeding of the bicarbonate ions to the biological culture in accordance with demands of the biological culture by retaining over-production of bicarbonate ions and feeding part of the over-production to the biological culture in accordance with nutrient demands of the biological culture. Bicarbonate ions may also be reconverted to generate a pure CO₂ gas stream. The CO₂ containing gas may be derived from operations of a power plant which receives a carbon-containing fuel for combustion, and the biological culture may be an algae culture.

### FIELD OF THE INVENTION

The present invention generally relates to the field of flue gas treatment with biological cultures such as algae cultures and, more specifically, to a system and process for enzymatic and metabolic conversion of CO₂ present in any gas into carbon containing bio-products.

### BACKGROUND

Treatment of CO₂ containing gas has in some cases used the enzyme carbonic anhydrase to enhance the hydration reaction of dissolved CO₂ into bicarbonate and hydrogen ions in an absorption solution. The absorption solution is then treated through precipitation or desorption in order to produce precipitated mineral solids or a relatively pure CO₂ stream for geologic sequestration or reutilization.

Biological cultures such as algae cultures have been generally recognized as an appropriate source of organic compounds such as pigments, biofuels, and feedstock for various applications.

However, known CO₂ capture methods and biological culture bio-production methods have a variety of drawbacks and disadvantages, for example in terms of efficiency, reliability and cost effectiveness. There is indeed a need in the industry for a technology that overcomes at least some of these drawbacks.

### SUMMARY OF THE INVENTION

Accordingly, the present invention responds to the above-identified need by providing a method, process, apparatus, use of carbonic anhydrase and formulation for dual biocatalytic conversion of CO₂ gas into carbon containing bio-products by enzymatic hydration of CO₂ into bicarbonate ions and metabolic conversion of the bicarbonate ions into carbon containing bio-products in a biological culture.

Captured CO₂, either as a mineral carbonate or pure CO₂ can be used to enhance the growth of biological cultures. Using carbonic anhydrase more efficiently provides biological cultures with the CO₂ carbon substrate for metabolism, resulting in overall greater process efficiency.

More specifically, in one aspect, the present invention provides a method for dual biocatalytic conversion of CO₂ in a CO₂ containing gas into carbon containing bio-products by enzymatically catalyzing the hydration reaction of dissolved CO₂ into bicarbonate and hydrogen ions in the presence of carbonic anhydrase and metabolically converting the bicarbonate ions into the carbon containing bio-products in a biological culture.

In an optional aspect, the method may include maintaining the dual biocatalytic conversion relatively constant and controlling a feeding of the bicarbonate ions to the biological culture in accordance with demands of the biological culture by retaining over-production of bicarbonate ions and feeding part of the over-production to the biological culture in accordance with nutrient demands of the biological culture.

In another optional aspect, the over-production of the bicarbonate ions may be retained in the form of carbonate precipitates.

In another aspect, the present invention provides a process for treating a CO₂ containing gas to produce carbon containing bio-products. The process includes:
a) contacting an aqueous absorption solution comprising water and an absorption compound with the CO₂ containing gas in the presence of carbonic anhydrase;
b) enzymatically catalyzing the hydration reaction of dissolved CO₂ into bicarbonate and hydrogen ions within the aqueous absorption solution to produce a bicarbonate loaded solution;
c) metabolically converting the bicarbonate ions in the bicarbonate loaded solution into carbon containing compounds by a biological culture; and
d) harvesting and treating the biological culture to extract the carbon containing compounds and transforming the same into the carbon containing bio-products.

In an optional aspect, the process may include controlling the temperature of the CO₂ containing gas before the step a) of contacting the aqueous absorption solution. Optionally, the process may include cooling the CO₂ containing gas before the step a) of contacting the aqueous absorption solution.

In another optional aspect, the process may include adjusting the pH of the aqueous absorption solution.

In another optional aspect, the process may include removing contaminants from the CO₂ containing gas before the step a) of contacting the aqueous absorption solution.

In another optional aspect, the process may include removing carbonic anhydrase from the bicarbonate loaded solution before the step c) of metabolically converting the bicarbonate ions.

In another optional aspect, the process may include recycling a portion of the bicarbonate loaded solution to make up the aqueous absorption solution before the step a) of contacting.

In another optional aspect, the process may include pre-treating the bicarbonate loaded solution before the step c) of metabolically converting the bicarbonate ions, to alter a solubility of the bicarbonate ions in the bicarbonate loaded solution to enhance precipitation thereof into carbonate precipitates. Optionally, the pre-treating may include altering the pH of the bicarbonate loaded solution and/or altering the temperature of the bicarbonate loaded solution. Optionally, the pre-treating may also include adding a cationic co-precipitating agent.

In another optional aspect, the process may include separating at least a portion of the precipitates, referred to as a precipitated solid fraction, from the bicarbonate loaded solution for downstream applications.

In another optional aspect, the process may include adjusting an amount of the precipitated solid fraction to be redistributed to the biological culture in accordance with monitoring growth cycles of the biological culture.

In another optional aspect, the process may include mixing the amount of the precipitated solid fraction to be redistributed with a liquid containing nutrients for the biological culture to form a supplemental bicarbonate nutrient stream for supply to the biological culture. Optionally, the liquid may be derived from a wastewater source.

In another optional aspect, the process may include pre-treating the liquid by chemical treatment, mechanical treatment, thermal treatment or a combination thereof. Optionally, the pre-treating of the liquid may include heating the liquid via a heat-exchanger to produce a pre-heated liquid.

In another optional aspect, the process may include desorbing CO₂ from at least a portion of the bicarbonate loaded solution and/or of the carbonate precipitates to generate a pure CO₂ gas stream and an ion-depleted solution recyclable as a portion of the aqueous absorption solution.

In another optional aspect, the process may include supplying the biological culture with various streams of nutrients, the nutrients comprising nitrogen compounds.

In another optional aspect, the process may include supplying light to the biological culture. Optionally, the light may be supplied continuously or intermittently, at a constant or variable intensity.

In another optional aspect, the step d) of harvesting and treating the biological culture may also produces a separated solution, and the process may include recycling a portion of the separated solution as the liquid containing nutrients to form the supplemental bicarbonate nutrient stream.

In another optional aspect, the process may include recycling a remaining portion of the separated solution to make up the aqueous absorption solution before the step a) of contacting.

In another optional aspect, the step c) of metabolically converting the bicarbonate ions may also produce a bicarbonate-depleted solution, and the process may include recycling at least a portion of the bicarbonate-depleted solution to make up the aqueous absorption solution before the step a) of contacting.

In another optional aspect, the step d) may include transforming the carbon containing compounds into bio-oils for lubrication, liquid fuels for energy supply, or a combination thereof. Optionally, the step d) may also include extracting biomass for use as solid fuel and/or feedstock. Optionally, the step d) may also include extracting a nutrient fraction to be supplied to the biological culture.

In another optional aspect, the process may include measuring and controlling a concentration and/or a flow rate of make-up streams which comprise an enzyme make-up stream, an absorption compound make-up stream, a solid precipitates make-up stream or a combination thereof, to make up the aqueous absorption solution.

In another aspect, the present invention provides an apparatus for dual biocatalytic conversion of CO₂ gas in flue gas into carbon containing bio-products. The apparatus includes:
an enzymatic bicarbonate production and CO₂ gas absorption unit including:
   a gas inlet for receiving the flue gas;
   a liquid inlet for receiving an aqueous absorption solution;
   a reaction chamber configured for receiving the flue gas and the aqueous absorption solution such for contact in the presence of carbonic anhydrase for catalyzing the hydration reaction of dissolved CO₂ from the flue gas into bicarbonate and hydrogen ions to produce a bicarbonate loaded solution and a treated gas;
   a gas outlet for releasing the treated gas; and
   a liquid outlet for releasing the bicarbonate loaded solution;
a biological culture unit comprising:
   an inlet for receiving the bicarbonate loaded solution;
   a culture compartment for metabolically converting the bicarbonate ions by a biological culture into carbon containing bio-products; and
   an outlet for releasing biological culture material containing the carbon containing bio-products from the culture compartment; and
an extraction unit for extracting at least some of the carbon containing bio-products from the released biological culture material.

In an optional aspect, the reaction chamber of the enzymatic bicarbonate production and CO₂ gas absorption unit may be a direct gas-liquid contact reactor. Optionally, the direct gas-liquid contact reactor may be a spray reactor, a packed bed reactor, a bubble reactor, a flow-wire reactor or analogs thereof.

In another optional aspect, the reaction chamber of the enzymatic bicarbonate production and CO₂ gas absorption unit may be an indirect gas-liquid contact reactor utilizing an enzymatic membrane for catalyzing the hydration reaction of the dissolved CO₂.

In another optional aspect, the apparatus may include a cooling unit, located upstream of enzymatic bicarbonate production and CO₂ gas absorption unit, receiving the flue gas and controlling the temperature of the flue gas so as to release a temperature controlled flue gas. Optionally, the cooling unit may be a heat exchanger receiving a cooling solution for controlling the temperature of the flue gas.

In another optional aspect, the apparatus may include a contaminant removal unit, located upstream of the enzymatic bicarbonate production and CO₂ gas absorption unit, for removing contaminants from the flue gas and produce a decontaminated flue gas, the contaminants comprising metals, SOx, NOx or a combination thereof. Optionally, the contaminant removal unit may be a scrubber receiving a scrubbing solution and releasing a contaminant-loaded solution containing nitrogen compounds.

In another optional aspect, the apparatus may include a treatment unit, located downstream the enzymatic bicarbonate production and CO₂ gas absorption unit, for altering a solubility of the bicarbonate loaded solution prior to enter the biological culture unit and form carbonate precipitates therein. Optionally, the treatment unit may include a separation device for separating at least a portion of the carbonate precipitates, referred to as a precipitated solid fraction, from the bicarbonate loaded solution for downstream applications. Optionally, the separation device may perform centrifugation, filtration, sedimentation or analogs thereof.

In another optional aspect, the apparatus may include a storage unit for holding the precipitated solid fraction before redistribution.

In another optional aspect, the apparatus may include a desorption unit, located downstream the treatment unit, receiving at least a portion of the bicarbonate loaded solution and/or of the carbonate precipitates for desorbing CO₂ and form a pure CO₂ gas stream.

In another optional aspect, the apparatus may include a solid-liquid mixing unit for mixing an adjustable amount of the precipitated solid fraction with a liquid containing nutrients to form a supplemental bicarbonate nutrient stream to be supplied to the biological culture unit. Optionally, the solid-liquid mixing unit may be an agitated tank.

In another optional aspect, the apparatus may include a nitrogen pre-treatment unit where the contaminant loaded solution is regenerated into the scrubbing solution and into a nitrogen nutrient stream to be supplied to the biological culture unit.

In another optional aspect, the apparatus may include a biological illumination unit to produce light to be supplied to the biological culture unit.

In another optional aspect, the culture compartment of the biological culture unit may include at least one photo-bioreactor. Optionally, the at least one photo-bioreactor may include several photo-bioreactors arranged in series and/or parallel, adjacent photo-bioreactors being connected to one another with open ponds, covered ponds or a combination thereof.

In another optional aspect, the apparatus may include a biological culture separation unit, located between the biological culture unit and the extraction unit, for separating a separated solution from the biological culture material. Optionally, the biological culture unit may include a solution outlet for releasing a bicarbonate-depleted solution.

In another optional aspect, the apparatus may include another separation device receiving the bicarbonate-depleted solution for removing any residual biomass from the latter.

In another optional aspect, the apparatus may include a pH adjustment unit, located upstream the biological culture unit, for adjusting the pH of a recyclable portion of the bicarbonate loaded solution which is used to make up the aqueous absorption solution.

In another optional aspect, the apparatus may include a measurement and control unit, located upstream the biological culture unit, to measure and control a concentration and/or a flow rate of make-up streams which comprise an enzyme make-up stream, an absorption compound make-up stream, a solid precipitates make-up stream or a combination thereof, to make up the aqueous absorption solution.

In another optional aspect, the extraction unit may include various chemical and/or mechanical extraction devices to produce bio-oils for lubrication, liquid fuels for energy supply, biomass for solid fuels and feedstock, a nutrient fraction for the biological culture or a combination thereof, from the released biological culture material.

In another optional aspect, the biological culture unit may be a first biological culture sub-unit and the apparatus may include a second or more biological culture sub-unit(s) operating in series or in parallel.

In another aspect, the present invention provides a use of carbonic anhydrase and a biological culture for sequential dual biocatalytic conversion of CO₂ gas in flue gas into carbon containing bio-products.

In another aspect, the present invention provides a use of carbonic anhydrase in a biological culture to accelerate the dissolution and conversion of CO₂ gas into bicarbonate and hydrogen ions for biological metabolism and conversion into carbon containing bio-products.

In another aspect, the present invention provides a dual biocatalytic formulation for conversion of CO₂ in a CO₂ containing gas into carbon containing bio-products, comprising water; CO₂ dissolved in the water; carbonic anhydrase in suspension in the water in sufficient amount to catalyze the hydration reaction of dissolved CO₂ into bicarbonate and hydrogen ions in the water in a nutritive bicarbonate concentration; and biological culture material in the water in sufficient amount to have sustained metabolic activity in the nutritive bicarbonate concentration for conversion of the bicarbonate ions into the carbon containing bio-products.

In another optional aspect, the aqueous absorption solution may include potassium or sodium carbonate in an amount sufficient to enhance CO₂ capture and/or to facilitate achieving controllable bicarbonate/carbonate concentrations. Optionally, the potassium carbonate may have a concentration between about 1M and about 2M, and wherein the sodium carbonate has a concentration between about 0.3M and 2.4M.

In another optional aspect, the aqueous absorption solution may have a temperature below about 30° C.

In another optional aspect, the pH of the aqueous absorption solution may be between about 8 and about 11.5.

In another optional aspect, the pH of the biological culture may be between about 7 and about 9.

In another optional aspect, the biological culture may be an algae culture.

In another optional aspect, the biological culture may produce at least part of the carbonic anhydrase for use in the enzymatic CO₂ capture.

In another optional aspect, the biological culture may include a micro-organism culture, such as cyanobacteria, e.g. Phormidium ambiguum, Phormirium orientalis, and/or Microcoleus sp., green algae, an alkaliphilic micro-organism culture, a halophilic micro-organism culture, an euglena culture, purple sulfur and non-sulfur bacteria culture, green sulfur and non-sulfur bacteria culture, nitrosomonas bacteria culture, nitrobacter bacteria culture, and/or methanogen archaea culture, and/or strains and variants and mixtures thereof.

In another optional aspect, the CO₂ containing gas may be derived from operations of a power plant which receives a carbon-containing fuel for combustion.

In another optional aspect, the carbonic anhydrase may be immobilized or entrapped on or in packing or internals of a reactor.

In another optional aspect, the carbonic anhydrase may be associated with free floating particles flowing through a reactor, the carbonic anhydrase being immobilized, bonded, entrapped and/or coated onto the particles using a stabilization material.

In another optional aspect, the carbonic anhydrase may be present as aggregates or crystals in suspension in an aqueous liquid.

In another optional aspect, the carbonic anhydrase may be dissolved and free in an aqueous liquid.

It should be understood that any one of the above mentioned optional aspects of each method, process, apparatus, use and formulation for dual biocatalytic conversion of CO₂ in a CO₂ containing gas into carbon containing bio-products, may be combined with any other of the aspects thereof, unless two aspects clearly cannot be combined due to their mutually exclusivity. For example, the various operational steps of the process described herein-above, herein-below and/or in the appended Figures, may be combined with any of the method, apparatus, use or formulation descriptions appearing herein.

### BRIEF DESCRIPTION OF THE DRAWINGS

FIG. 1 is process block flow diagram of an embodiment of the process of the present invention.

FIG. 2 is a process flow diagram of another embodiment of the process of the present invention.

FIG. 3 is a graphic of fractional amounts of carbonic acid, bicarbonate and carbonate in relation to pH of the solution.

FIG. 4 is a process block flow diagram of another embodiment of the process of the present invention.

FIG. 5 is a process block flow diagram of another embodiment of the process of the present invention.

FIG. 6 is a process block flow diagram of another embodiment of the process of the present invention.

### DETAILED DESCRIPTION OF THE INVENTION

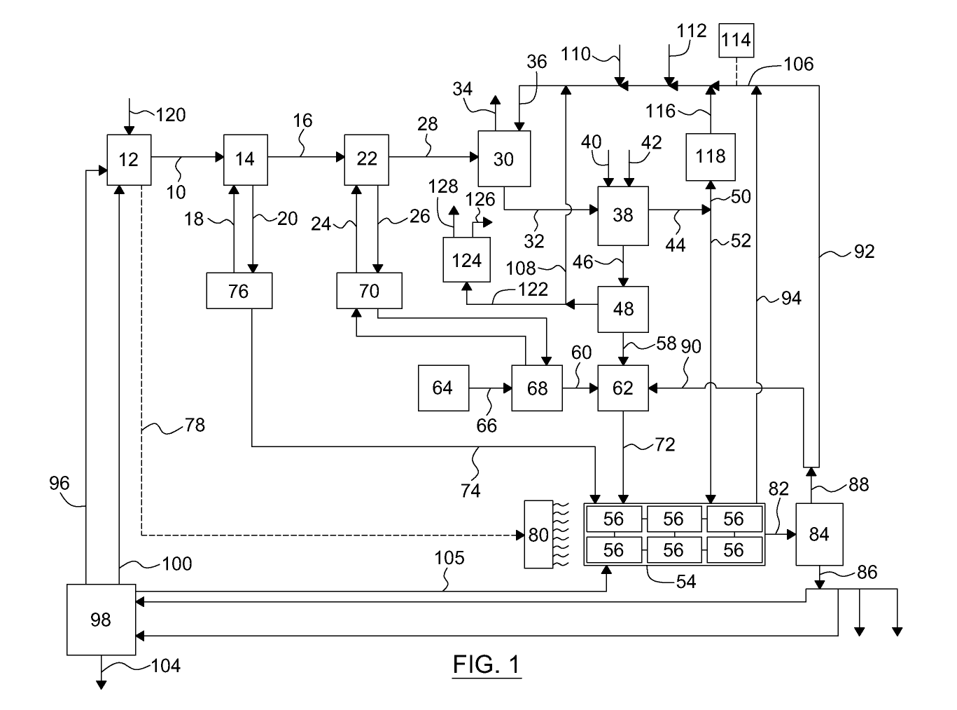

Referring to FIG. 1, CO₂ containing gas 10 is generated in from a gas source 12. The CO₂ containing gas 10 may be flue gas or another type of gas from an industrial or power plant or any other CO₂ emitting source. The CO₂ containing gas 10 that is processed may be a portion or slip stream of the overall emitted gas from the gas source 12. The CO₂ containing gas 10, which may also be referred to herein as “flue gas”, is optionally fed to a contaminant removal unit 14. The removal unit 14 is for removing contaminants such as metals, SOx and/or NOx compounds from the flue gas 10 and thereby producing a decontaminated flue gas 16. The removal unit 14 will of course depend on the type of CO₂ containing gas 10 to be treated. The contaminant removal unit 14 may be a scrubber receiving a scrubbing solution 18 which may be sprayed or otherwise provided to the scrubber. A contaminant-loaded solution 20 is removed from the scrubber 14 and contains various contaminants depending on the composition of the flue gas 10.

The decontaminated flue gas 16 is then provided to a cooling unit 22, which removes heat from the hot flue gas 16. In some aspects, the flue gas 10 may not be hot enough to merit cooling and the cooling unit 22 may therefore be considered as optional. In other aspects, the cooling unit is a heat exchanger which may receive a cooling fluid 24 which receives heat from the hot flue gas 16 and becomes a heated exchanger fluid 26. A cooled CO₂ containing gas 28 is therefore produced. This gas may also be referred to as a temperature controlled CO₂ containing gas 28, as its temperature is preferably controlled to be sufficiently low for downstream process steps as will be described below.

Referring still to FIG. 1, the cooled CO₂ containing gas 28 is provided to an enzymatic bicarbonate production unit 30. The enzymatic bicarbonate production unit 30 comprises a reactor or absorber in which the CO₂ undergoes an enzymatically catalyzed hydration reaction into bicarbonate and hydrogen ions in aqueous form, thereby producing a bicarbonate-loaded solution 32, which will also be referred to as a loaded absorption solution 32. The enzyme catalyzes the reversible reaction CO₂+H₂O←→HCO₃⁻+H⁺. A CO₂ lean gas 34 is released from the bicarbonate production unit 30. In one aspect, bicarbonate production unit 30 receives an absorption solution 36 for absorbing the CO₂. In one preferred aspect, the absorption solution 36 and the CO₂ containing gas 28 contact each other directly within the reactor of the bicarbonate production unit 30. Such direct gas-liquid contact reactor may be a spray reactor, packed bed reactor, bubble reactor, flow wire reactor, or another type of reactor design. In an alternative embodiment, the reactor may be an indirect contact gas-liquid reactor utilizing an enzymatic membrane for the CO₂ capture. The CO₂ from the flue gas is thus trapped in the loaded absorption solution as carbonates/bicarbonates.

The loaded absorption solution 32 may then be provided to a treatment unit 38 for pre-treating the solution prior to integration with downstream biological cultures and for managing bicarbonate inventories. In one aspect, the treatment unit 38 may alter the solubility of the bicarbonate and carbonate ions present in the loaded absorption solution 32. This may be accomplished by altering the pH and/or temperature of the solution. A pH adjusted stream 40 and/or a temperature adjusted stream 42 may be provided.

The loaded absorption solution 32 is also optionally treated and divided into at least two separate streams, a diluted or concentration-controlled bicarbonate solution 44 and a precipitated solid fraction 46. The solid fraction 46 may be provided further treated or processed (drying for example) prior to being provided to a storage unit 48 for holding until needed or for redistribution to different biological cultures, markets and applications on other sites. The concentration-controlled bicarbonate solution 44 may also be split in certain optional aspects of the process. For example, the solution 44 may be split into a direct recycle component 50 and a biological feed component 52, which is also referred to as a bicarbonate nutrient solution 52. In a case where the biological culture site is not proximate, the loaded absorption solution may be transported by pipeline and the solids/precipitates may be removed and stocked proximate the biological culture site, thereby avoiding transport by trucks or train. If more than one biological culture is provided to treat the loaded absorption solution, the latter may be sent to several cultures in parallel or in series.

The loaded absorption solution 32 or a portion thereof may be provided directly to the biological culture, as a pure liquid solution or as a slurry containing solids.

The bicarbonate nutrient solution 52 is fed to a biological culture unit 54. The biological culture unit 54 may include one or several photo-bioreactors (PBR) 56 or tanks, or open and/or covered ponds, arranged in series and/or parallel. The bicarbonate nutrient solution 52 provides a carbon source to the biological culture for promoting advantageous growth in an efficient manner, as will be further described below.

In one optional aspect, the stored carbonate solids 46 may be used to supplement the biological culture unit 54 at times of increased bicarbonate demand during the growth cycle or other times depending on process parameters. A portion of carbonate solids 58 may be mixed with a liquid 60 in a mixing unit 62 which may be a tank that is agitated or not or another type of solid-liquid mixing unit. It should also be noted that the carbonate solids 46 and 48 may be transported and stored in the form of a slurry containing some liquid and the mixing unit 62 may therefore be provided to mix the slurry with additional water 60. The additional water 60 may be derived from a wastewater source 64, for example. Depending on the biological culture nutrient demands, the water 66 may be obtained from an appropriate source.

Wastewater can provided a good source of nutrients for the biological culture, notably during preparation of the biological cultures or during periods of high nutrient demand. The wastewater stream 66 may be fed to a pre-treatment unit 68 which may provide a chemical, mechanical and/or thermal pre-treatment. It may be preferred to heat the stream 66, which may be accomplished through integrated reuse of heat derived from the cooling unit 22. For instance, the heated fluid 26 may be used directly or via a heat exchanger 70 to heat the stream 66 and produce a pre-heated water 60 which thus facilitates dissolving the carbonate solids and/or slurry 58 in the mixing unit 62. The mixing unit 62 thus produces a supplemental bicarbonate nutrient stream 72 for introduction into the biological culture unit 54.

In some optional aspects of the present invention, the biological culture unit 54 may receive additional input streams of various types. Referring still to FIG. 1, the system optionally has various streams and units for energy and fluid interaction and integration between the flue gas treatment and the biological culture.

For example, in one aspect, there may be a nitrogen containing stream 74 that is provided to the biological culture unit 54 for supplying a nitrogen source. The nitrogen containing stream 74 may be at least partially derived from the nitrogen containing compounds scrubbed out of the flue gas 10, thereby further integrating the flue gas treatment with the biological culture. The contaminant-loaded solution 20 containing nitrogen compounds would be supplied to a nitrogen pre-treatment unit 76, which may also be referred to as scrubbing liquid regeneration unit, where the contaminant-loaded solution 20 is treated preferably to regenerate the scrubbing solution 18 for reuse in the scrubber 14 and also to recuperate compounds from the contaminant-loaded solution 20 and provide them in the form of nutritive components within additional nutrient stream 74 which may preferably be a nitrogen containing stream.

Referring still to FIG. 1, in another optional aspect, a portion of the electricity 78 generated by the combustion of fossil fuels in the power plant 12 is provided to power at least one biological illumination unit 80. Of course, this optional aspect is for the embodiment where the gas is flue gas from a power plant and would not apply if the CO₂ comes from certain other sources. This electricity may be provided directly from the power plant 12 or indirectly through the grid. The biological illumination unit 80 may be operated continuously, at a constant or variable intensity, or intermittently when a natural solar light is unavailable.

Portions of some streams that are output from the biological culture may also be reused as components in the input streams, as will be further described below.

Referring still to FIG. 1, the biological culture unit 54 includes an outlet for withdrawing a biological culture harvest stream 82 in the form of a slurry containing some liquid and biological culture. The biological culture harvest stream 82 is provided to a biological culture separation unit 84. The biological culture separation unit 84 produces at least harvested biological culture 86 and a separated solution 88. The biological culture separation unit 84 may separate the biological culture harvest stream into more than two streams.

The separated solution 88 may be split such that a portion of it is used, for example, as an aqueous solution 90 provided to the mixing unit 62 for dissolving the carbonate solids and/or slurry 58 for producing the supplemental bicarbonate nutrient stream 72.

The separated solution 88 or at least a substantial portion thereof is preferably recycled to make up a portion 92 of the regenerated solution for use as the absorption solution 36. The biological culture unit may have a solution outlet through which a bicarbonate-depleted solution 94 is withdrawn for recycling as another portion of regenerated solution for use as the absorption solution 36. The bicarbonate-depleted solution 94 should pass through a filter or another separation device to remove any residual biomass or unwanted material from the solution 94 which could foul or form a biofilm in the bicarbonate production and CO₂ capture unit 30. It should be understood that there may be a number of streams that may be generally referred to as bicarbonate-poor or microbially regenerated streams, which are treated and/or combined for eventual recycling as at least part of the absorption solution 36. A substantial portion of the absorption solution is therefore recycled throughout the process, the solution being supplemented by water, enzyme, biological culture and nutrients if needed. It should therefore be understood that the biological culture may act as a CO₂ capture regeneration unit and after regeneration in the biological culture the carbonate solution is sent back to the enzymatic bicarbonate production and CO₂ capture unit 30.

Referring still to FIG. 1, there may be additional heat and fluid integration between the flue gas treatment and the biological culture. For example, the harvested biological culture harvested 86 may be processed and converted into a number of different useful products that may be reused in the system. In one optional aspect, the harvested biological material 86 is processed to produce dried residual low-value biomass 96, for example by providing the harvested biological material 86 to an extraction unit 98 which extracts high-value products leaving the residual biomass 96. This residual biomass may be used as a source of combustible fuel in the power plant, thereby further recuperating and utilizing the solar, electric and heat energy of the biological material. In another optional aspect, the harvested biological material 86 is processed to produce lubricant 100 for equipment in the power plant 12. Bio-oils that are appropriate for equipment lubrication may be derived from the harvested biological material in the extraction unit 98, which may include various chemical and/or mechanical extraction devices. The extraction unit 98 may also produce liquid fuels 104, such as biodiesel and/or bioethanol, a part of which may be used in the power plant 12 machinery. Algae biomass may also be prepared for direct use as a solid fuel. In addition, biomass may be at least partly or fully dried using heat recovered at the heat exchanger 22. The extraction unit 98 may also produce recyclable extracted nutrient fraction 105 that may be fed back into the biological culture unit 54.

Referring still to FIG. 1, in some optional aspects of the present invention, various regenerated streams are combined and provided as an overall regenerated solution 106. The overall regenerated solution 106 may be composed of streams 92 and/or 94 from the biological culture. The overall regenerated solution 106 may be pre-treated to remove any undesirable components prior to reuse as the absorption solution 36. In addition, various streams may be added to the overall regenerated solution 106 in order to provide desired concentrations of certain components. For example, a recycled stream such as a solid precipitates make up stream 108 from the storage unit 48 may be added to the overall regenerated solution 106 to adjust the carbonate concentration. In addition, an enzyme make up stream 110 may be added to increase the enzyme concentration to aid catalysis in the bicarbonate production unit 30. Furthermore, an absorption compound make up stream 112 may be added to the overall regenerated solution 106 to adjust the concentration to aid absorption in the bicarbonate production unit 30. The absorption compound may be sodium and/or potassium carbonate or any other compound compatible with both CO₂ absorption and culture growth. In some cases, compounds such as ammonium carbonate, amines, alkanolamines (e.g. MDEA), amino acids, or different salts than potassium and sodium such as lithium and calcium, etc., may be used. The absorption compound make up stream 112 may be controlled in accordance with a set or desired carbonate concentration. There may be a measurement and control unit 114 for measuring or monitoring concentrations and/or flow rates of the overall regenerated solution 106 and for controlling the make-up doses of streams 108, 110, 112 and the like that are added to the overall regenerated solution 106 to produce a constant absorption solution 36. There may also be a liquid bicarbonate/carbonate recycle stream 116 that is added to the overall regenerated solution 106 and which is controlled by the control unit 114. The liquid bicarbonate/carbonate recycle stream 116 may be derived from a portion of the direct recycle component 50 of the loaded bicarbonate solution 44. The direct recycle component 50 may be pre-treated in a pH adjustment unit 118 prior to combination with the overall regenerated solution 106, in order to balance the bicarbonate and carbonate ions to give the desired proportion in the absorption solution 36.

Turning to the plant 12 that produces the flue gas 10, it should be noted that it may be any number of flue gas producing installations. In one preferred embodiment, the plant 12 is a power plant which receives a carbon-containing fuel 120 for combustion. The carbon-containing fuel may be fossil fuel such as coal, coke, solid or liquid petroleum or natural gas, or biomass fuel such as wood, plant matter biofuel or biogas which may be provided in various forms such as solid pellets as well as liquid or gas streams.

Referring still to FIG. 1, in some optional embodiments, at least a portion of bicarbonate/carbonate that has been removed from the flue gas is provided to an alternative regeneration or treatment unit. For example, at least a portion of the loaded solutions 32 or 44 or the precipitated solid/slurry 46 may be provided as an input stream 122 to a desorption apparatus 124 for desorbing CO₂ and producing a pure CO₂ gas stream 126 and an ion depleted solution 128. The ion depleted solution 128 may be recycled to form part of the absorption solution 36. This desorption treatment may be provided particularly if the biological culture unit has periods when it does not require bicarbonate nutrients (e.g. during maintenance or turn down phases, for example) while the enzymatic bicarbonate production unit 30 continues generating high quantities of captured CO₂. Since flue gas 10 production is continuous and it is desirable to continuously capture CO₂ gas and produce bicarbonate, it is preferred that the bicarbonate loaded solution 32 be continuously regenerated to enable recycling back into the enzymatic unit 30. Therefore, the regeneration strategy may alternate or be modified to adjust to the regeneration capacity of the biological culture unit 54. The additional product stream of pure CO₂ gas 126 is also a marketable and usable product.

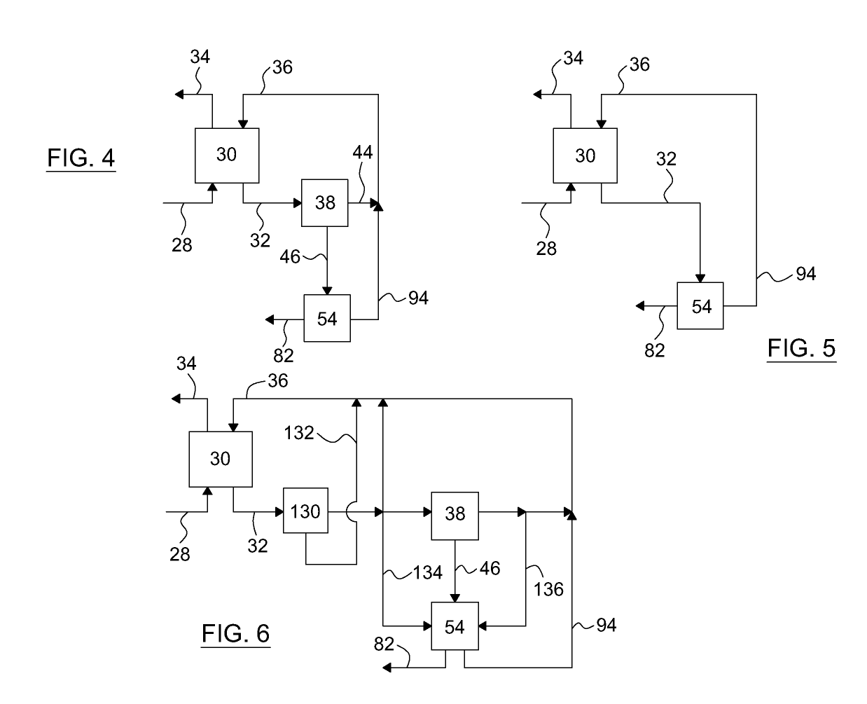

FIGS. 4-6 illustrate various embodiments of integrating the capture unit 30 with the biological culture unit 54. In FIG. 4, bicarbonate solid 46 is produced and fed to the biological culture unit 54 as a solid, and the separated liquid 44 is combined with the separated bicarbonate-depleted solution 94 to form the recycled absorption solution 36. In FIG. 5, the ion-rich stream 32 is fed as a solution or slurry, without separation, directly into the biological culture unit 54. As shown in FIG. 6, there may be several different ways of providing the bicarbonate nutrients to the biological culture unit 54, including a combination of solid and liquid streams. There may be other separation devices 130, for example for removing enzyme particles that may be used in the capture unit 30 and are removed from the ion rich solution 32 prior to downstream processing. The separated particles 132 may be returned to the absorption solution 36. Ion-rich addition stream 134 may be used to feed the biological culture unit 54 with bicarbonate nutrients and water while an ion-poor addition stream 136 may be used to add, clean or dilute the biological culture unit 54 if necessary.

Various aspects and embodiments of the process and system of the present invention will be further described below.

Flue gas that is rich in CO₂ is treated in a bicarbonate production and CO₂ capture system enhanced by the enzyme carbonic anhydrase or analog thereof. The CO₂ in the flue gas is dissolved and trapped in a carbonate/bicarbonate solution. The carbonate/bicarbonate solution or a precipitated carbonate/bicarbonate solid derived from the carbonate/bicarbonate solution, is sent to a biological culture as a source of carbon nutrients to promote biological growth. The carbonate/bicarbonate is supplied to the biological culture and is essentially stripped from the solution by the biological culture. The stripped solution is sent back from the biological culture to the enzymatic bicarbonate production and CO₂ capture system as a regenerated absorption solution. Biological material is harvested and may be transformed into high value products such as specialty chemicals, biofuels, plastics, pigments, feedstock, biomass, nutraceuticals and the like.

### Enzymatic Bicarbonate Production

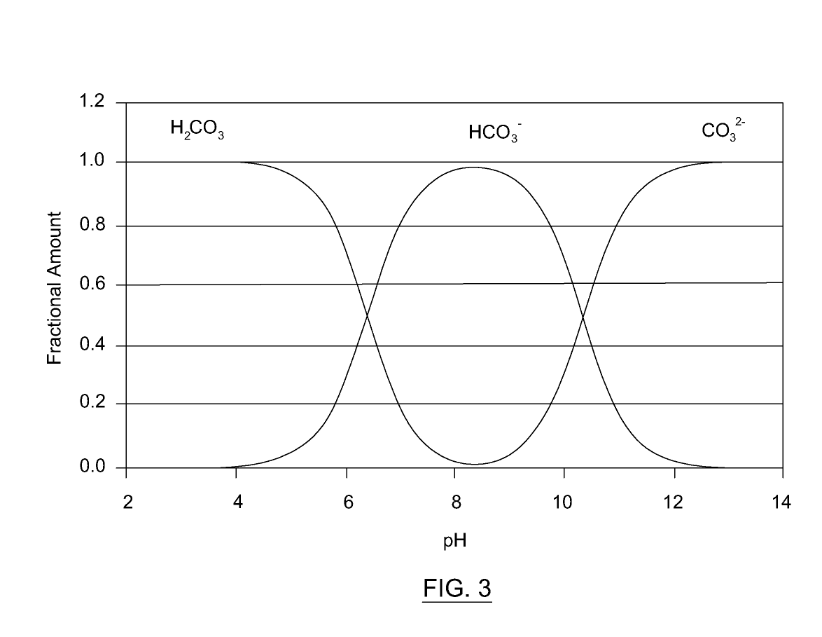

Flue gas emitted by a plant, such as a power plant, cement plant or other CO₂ emitting installation, is first treated according to regulations that may be in effect in a given jurisdiction to remove contaminants such as metals, SOx, NOx, and the like. The treated flue gas is then provided to the CO₂ capture unit. Additional gas cooling may be desirable or required prior to the CO₂ capture unit, depending on the desired processing parameters and the temperature resistance of the carbonic anhydrase that will be used. Residual NOx, if any, present in the contaminant treated flue gas, could also eventually be sent to the biological cultures as a source of nitrogen, if well absorbed into the solution. The treated flue gas passes through the CO₂ capture unit, which is preferably operated to remove about 90% or more of the CO₂ contained in the original gas. It should be noted that the removal may be adjusted to be below or above 90%. The CO₂ scrubbed gas is then released from the CO₂ capture unit, for example into the atmosphere. The CO₂ capture unit may include various different kinds of reactors, including a bubble reactor, packed bed column, spray tower, or another type of reactor, provided it uses an absorption solution into which the CO₂ is absorbed and that can be sent as bicarbonate feed solution to the biological cultures. In one preferred aspect of the present invention, the CO₂ capture unit uses an absorption solution comprising sodium and/or potassium carbonate. The absorption solution stocks CO₂ as bicarbonate and/or carbonate, depending on the pH of the solution. In this regard, FIG. 3 shows the relationship between bicarbonate, carbonate and carbonic acid according to pH.

Above certain concentrations, the bicarbonate in solution will start forming precipitates. The CO₂ capture unit may be operated to avoid such precipitates or to allow precipitation or even favor it. Avoiding precipitation simplifies treatment and handling of the absorption solution as it flows through the reactor, while precipitation may be helpful when additional carbonates are required for biological growth or are desirable for stocking while waiting for the biological culture to be available to treat it. Precipitation may also be helpful if installing an alternate regeneration system for the solution is envisaged and/or if transportation to another site for culture growth or other applications is desired.

The absorption solution that is fed to the enzymatic bicarbonate production unit may be a sodium carbonate solution having a sodium carbonate concentration between about 0.3M and about 2.4M (temperature being between about 30 t for a concentration of about 2.5M) or a potassium carbonate solution having a potassium carbonate concentration between about 1M and about 2M. Tests have confirmed that carbonic anhydrase has good activity in sodium carbonate between 0.3M and 0.5M and in potassium carbonate at 1.45M. Halophile-type carbonic anhydrase, with elevated resistance to salt, may be used for higher concentrations. As opposed to carbonates, bicarbonates are less soluble (two times less approximately) and, therefore, if precipitation occurs, it would be of bicarbonate salts rather than carbonate salts. Cooling the ion loaded solution exiting the enzymatic CO₂ capture unit may also encourage precipitation, as the solubility of bicarbonates and carbonates would be lowered. For sodium bicarbonate, temperature may be carefully adjusted since below about 30° C., a solution at 2.5M would not be soluble. The following Solubility Table may be used to set, adjust or control the carbonate or bicarbonate concentration as well as temperature in the enzymatic bicarbonate production and CO₂ capture unit:

Solubility Table

| | Temperature | Solubility |
|---|---|---|
| Compound | (° C.) | (M) |
| Na₂CO₃ | 20 | 1.7 |
| | 30 | 2.7 |
| | 40 | 3.1 |
| | 50 | 3.0 |
| | 60 | 2.9 |
| NaHCO₃ | 20 | 0.8 |
| | 30 | 0.9 |
| | 40 | 1.0 |
| | 50 | 1.2 |
| | 60 | 1.3 |
| K₂CO₃ | 20 | 4.9 |
| | 30 | 5.0 |
| | 40 | 5.1 |
| | 50 | 5.1 |
| | 60 | 5.2 |
| KHCO₃ | 20 | 2.3 |
| | 30 | 2.6 |
| | 40 | 2.9 |
| | 50 | 3.2 |
| | 60 | 3.5 |

Note:
Data in wt % transformed in Molar.

As mentioned above, the bicarbonate production and CO₂ capture unit is enhanced by the use of carbonic anhydrase. Thus, the enzymatic bicarbonate production unit employs carbonic anhydrase to capture CO₂ and produce a loaded bicarbonate solution. The carbonic anhydrase may be (a) immobilized or entrapped on or in packing or internals of the reactor, (b) associated with free floating particles flowing with the solution through the reactor (immobilized, bonded, entrapped and/or coated onto the particles using an stabilization material), (c) present as aggregates or crystals (CLEAs or CLECs) in suspension in the liquid, (d) or may be dissolved and free in solution. It should be noted that the enzymes may be associated with particles, packing or internals of an absorption reactor in any way that allows the enzyme to be available to catalyze the desired reaction. The carbonic anhydrase increases the bicarbonate production and CO₂ capture efficiency of the unit. The concentration of bicarbonate/carbonate captured in solution may be controlled with the capture solution concentration, the enzyme concentration and/or operating parameters of the unit. A higher concentration of bicarbonate/carbonate can be obtained more easily with the use of carbonic anhydrase, thus diminishing the volume or circulation flow rates required of the solution.

When there is no precipitation, the loaded solution may be simply sent to the biological cultures as a nutrient supply stream. When precipitation does occur, the loaded solution may be fed to the biological cultures as a slurry or the precipitated solids may be recovered as a particulate or powder material to be stored for later use, for example when additional bicarbonate/carbonate are wanted. The amount of precipitated solids that are fed to the biological culture may be adjusted in accordance with monitoring the growth cycles of the biological culture, for example. A co-precipitating agent, such as cations like calcium for example, may be used if precipitation is desired. Cooling the loaded solution would also favor precipitation as compounds would become less soluble in the liquid. Different techniques can also be used to recover the precipitates, such as centrifugation, filtration, sedimentation and the like.

The biological culture removes at least part of the bicarbonate/carbonate from the nutrient loaded solution fed to the culture. After removal of part of the bicarbonate/carbonate by the biological culture, the solution is sent back to the enzymatic bicarbonate production and CO₂ capture unit as a recycled absorption solution to further absorb CO₂ in the reactor. The reaction in the biological culture should therefore be provided, adjusted and/or controlled, so as to regenerate the solution at or proximate to its starting concentration of carbonates, and not to deplete it entirely of all carbonates.

In one example, in the absorption process the following reaction occurs:

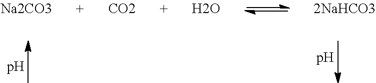

This reaction will often transform most of the carbonates, but not all, since obtaining saturation in bicarbonate is usually not cost effective.

In the biological culture the following reaction occurs:

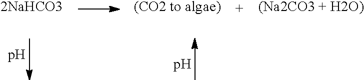

The biological culture may use part of the sodium and/or potassium from the loaded solution, as well as an elevated quantity of the carbonate. Consequently, it may be preferred to supplement the regenerated absorption solution which is fed back to the enzymatic bicarbonate production and CO₂ capture unit to bring it back to its original concentration or sodium and/or potassium carbonate levels before reusing it in the CO₂ absorption process.

In the biological culture: 2NaHCO3→1NaHCO3 to algae+1NaHCO3

After treatment by the biological culture: NaHCO3+NaOH→Na2CO3+H2O

In an optional aspect, in the Na₂CO₃/NaHCO₃ system the pH in the absorber will start at around 11.5 and go to 8-9. The 8-9 solution can be fed to culture without pH change, although it may be modified if desired for a particular culture or depending on the particular outlet solution pH. The culture will then grow and, in appropriate conditions, the pH will return to about 11.5. However, pH adjustment may be desired or necessary before returning the solution to the absorber, depending on the operating conditions of the process. 2NaHCO3→1NaHCO3 to algae+1NaHCO3

In another optional aspect of the present invention, the solution may be restored to its initial concentration by addition of some of the precipitates recovered during the process as discussed above. The composition of the carbonate/bicarbonate solution is a question of equilibrium between the species (carbonate and bicarbonate ions), which changes depending on factors such as pH. At high pH, most bicarbonate would transform into carbonate. FIG. 3 illustrates the various equilibrium aspects between carbonate and bicarbonate at different pH levels.

### Biological Culturing Unit

Depending on the relative capacities of the enzymatic bicarbonate production unit and the biological culture unit, several biological culture sub-units which may be ponds and/or photo-bioreactors and/or or tanks (tanks and containers may allow light passage or not depending on the organism; for instance, in the case of non-photosynthetic organisms such as methanogen archae, one would use a container but no light) may be preferred and configured in parallel or in series. If one biological unit cannot treat all of the loaded solution, a second unit or more may be installed in parallel. If all the loaded solution may be treated but not to a sufficient level to be recycled to the CO₂ capture unit, a second system or more may be preferred and provided in series to remove the desired level of ions for regeneration.

To maintain consistent treatment of the loaded bicarbonate solution, at least two culture sub-units is preferable, in order to allow a switch from one to the other when one has to be taken offline for maintenance, resulting from regular operation or contamination of the culture. In absence of a second culture sub-unit, bicarbonate solution would have to be treated by an alternate regeneration system like a desorption unit or stocked until a biological culture can treat it again.

Biological culture units may be in the form of ponds or photo-bioreactors or tanks. In one embodiment, the biological culture comprises micro-organisms. A photo-bioreactor with a micro-organism strain able to grow under constant illumination may be preferable as there would be no need to interrupt the feeding process for the night or dark period. If the biological cultures are not constantly growing because of the illumination cycles, at least two cultures with opposite illumination cycles may be desirable to ensure constant treatment of the bicarbonate solution.

If constant treatment of the bicarbonate loaded solution is not provided by the biological culture units, for example if sunlight is used and the non-photosynthetic period is the same for all cultures, an alternative treatment may be provided or the solution may be stocked until further processing. In the first case, carbonate could be stocked as precipitate, with the solution being regenerated mostly through the precipitation process. In the second case, it may be preferable that the biological cultures, when in operation, have a larger regeneration capacity than the flow of carbonate solution, to be able to treat backlogged solution. This could be true even for one of the alternative regeneration embodiments, as when precipitation is used, the precipitate could be fed to the biological culture.

In one preferred aspect of the present invention, the biological culture unit utilizes micro-organisms such as “micro-algae”. In one embodiment, the micro-organisms are green micro-algae and/or cyanobacteria. The micro-organism strain may be selected for use in high concentrations of bicarbonates to further increase efficiency of the process, as a more concentrated carbonate solution would capture more CO₂. The typical pH for micro-organism growth is 7 to 9. Higher than that, the use of an alkaliphilic strain of micro-organism would be desirable. Such strains, mostly cyanobacteria, grow at pH 9.5 to 10.5, with carbonate concentration of 1 to 2.5M, depending of the strains. Among cyanobacteria, alkaliphilic strains as reported in the literature are Phormidium ambiguum, Phormirium orientalis, Microcoleus sp., which can grow at pH 9.5 to 10.4, with about 1M of sodium carbonate. Eukaryotic green algae have been isolated from a soda lake, which can grow at pH 10.2 with between about 2M and about 2.5M of sodium carbonate.

It should also be noted that other kinds of organisms and algae can be employed to use CO₂, carbonate and/or bicarbonate as a substantial source of carbon. Such organisms include and are not limited to the following: algae, some cyanobacteria, euglena, purple sulfur and non-sulfur bacteria, green sulfur and non-sulfur bacteria, nitrosomonas bacteria, nitrobacter bacteria, methanogen archaea, including strains and variants thereof, etc. Most of such organisms use light as their main source of energy. But some use inorganic compounds as a source of energy, such as ammonia, sulfur, hydrogen, etc. The latter organisms would require a constant supply of chemical instead of light. In one aspect, the biological culture is fed with H₂ which may also be derived from an industrial H₂-generating source which may be the same or different from the CO₂-generating plant 12. A biological culture can be made of a pure unique culture or a combination of several different kinds of organisms. A combination culture contains at least one kind of organism able to fix CO₂ (or carbonate or bicarbonate). The other organisms can be any organism, preferably that promotes or enhances the CO₂ fixing culture or whose growth and bio-product production is promoted or enhanced by the presence of the CO₂ fixing culture. Having a mixture of different organisms may lead to a greater CO₂ usage capacity or different and/or more valuable end products.

In another optional aspect of the present invention, micro-organism strain capable of carbonic anhydrase secretion may be used. In this aspect, carbonic anhydrase that is secreted may be sent back to the CO₂ capture system with the de-carbonated solution, thus providing an internal source of free enzyme to catalyze the CO₂ absorption reaction. Carbonic anhydrase would not be naturally expressed at high carbonate concentrations, but secretion may be promoted once the biological culture reached high density. Genetic manipulation of micro-organisms may also be used to provide a strain that is genetically modified to produce or over-produce carbonic anhydrase, and thus carbonic anhydrase expression may be enhanced and made more constant in the process. The presence of carbonic anhydrase in the biological culture should not cause problems. It should also be noted that biological cultures such as micro-algae usually do not express carbonic anhydrase at high bicarbonate concentrations, not because it is harmful to the micro-algae, but rather because it is not required. In the case where the given micro-algae or other micro-organism strain would secrete proteases that may be harmful to free carbonic anhydrase, an immobilized or stabilized carbonic anhydrase in the absorber and/or on or in particles may be used. The recovered biological material could be used to produce biofuels, feedstock for fish, oysters and the like, pigments or any other valuable product the strain would be suitable for.

In order to prepare and maintain the biological culture medium, wastewater may be used as a source of nutrients, since it may be rich in minerals and a good source of nitrogen and phosphate. Enough nutrients should be provided to ensure appropriate biological growth and, therefore, addition of nutrients may be desirable. Continuous nutrient addition may be preferred. It is also possible to simply prepare a biological culture growth medium that is generally known in the art.

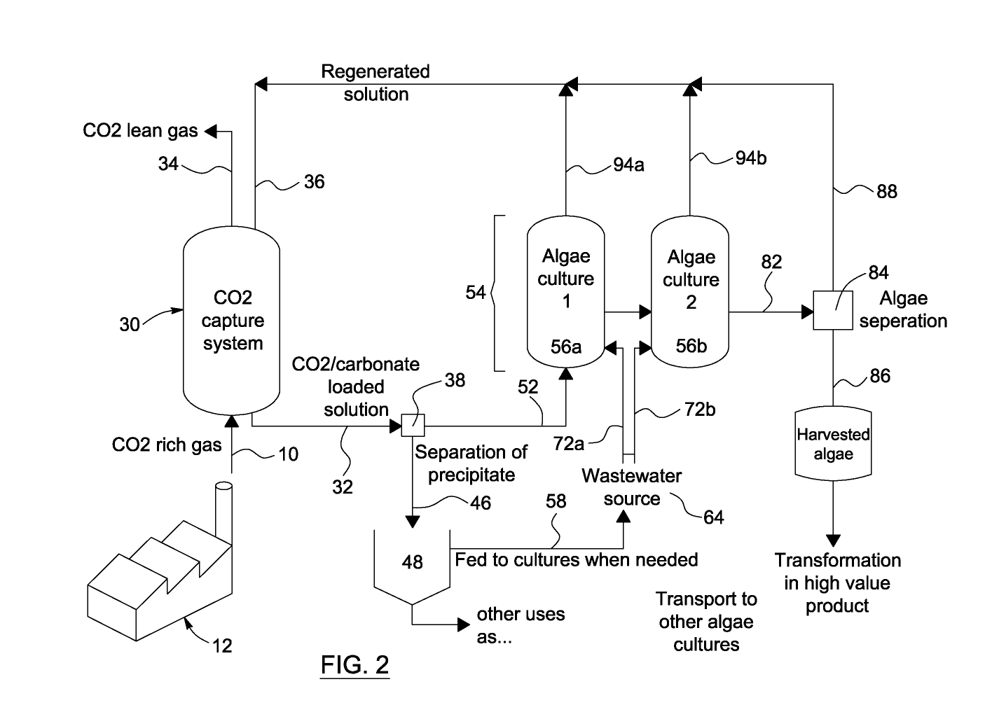

If required, the carbonate solution recovered from a first biological culture sub-unit 56a may be directed to a second biological culture sub-unit 56b for further carbonate fixation by the biological culture and so on. Such units may also receive two different streams of wastewater 72a, 72b and may produce two streams of bicarbonate depleted solution 94a, 94b, as shown in FIG. 2. Once the solution is de-carbonated to a satisfactory level, it may be sent back to the enzymatic bicarbonate production and CO₂ capture unit 30. Different biological strains may be used in each biological culture unit or in each overall system according to the varying levels of carbonates. In this case, it could be preferred to filter the solution between the biological culture units, in order to avoid contamination from one culture to another.

Harvesting biological culture material may be performed in various ways, such as harvesting the whole culture before starting a fresh one (batch cultures); continuously harvesting part of the culture in line with the growth rate (continuous culture); or a mix of these two strategies. A mixed strategy would include periodically harvesting part of the culture and adding new solution to continue the growth. Continuous cultures are preferable for the present invention.

In one optional scenario, the process includes the addition of carbonic anhydrase and CO₂ directly into the biological culture unit for in situ conversion of the carbon dioxide into bicarbonate within the biological culture unit. The biological culture unit may be equipped with carbon dioxide bubble injector and an inlet for providing the carbonic anhydrase in the form of a solution or a solid or in a particular form as desired. The biological culture unit may also be equipped with agitation or fluid flow mechanisms for encouraging mass transfer while avoiding biological culture damage, thereby promoting the conversion of carbon dioxide into bicarbonate for culture metabolism. During harvest, the culture biomass is preferably removed from the liquid containing the carbonic anhydrase, which is retained for subsequent biological culture production. The CO₂ containing gas may be pretreated in accordance with the metabolic capabilities and toxicity related to the particular biological culture, and the gas may be directly supplied to the culture as a mixed gas or the CO₂ containing gas may be enzymatically processed to generate a pure CO₂ stream that is supplied to the biological culture. For example, carbonic anhydrase may be provided in an algae pond or photobioreactor which receives CO₂ as a carbon source which is converted into bicarbonate ions within the bioreactor in an accelerated manner. This scenario may also be combined with one or more of the embodiments of the process and system described and illustrated herein.

A variety of CO₂ containing gas types may be processed by embodiments of the present invention into bio-products. In some embodiments, the CO₂ emitting source also emits or produces a chemical stream which is also useful in the biological culture, as a nutrient or energy source for example, to promote biological culture growth. Nitrogen or hydrogen containing streams, for instance, may be useful as a nutrient or energy source for certain biological cultures. The biological culture therefore consumes waste gas directly in presence of carbonic anhydrase or after a separate enzymatic pre-treatment step for isolating CO₂ gas or preparing a bicarbonate stream. While the integrated process may benefit from proximal locations of the enzymatic CO₂ capture unit and the biological culture unit, it is also possible to transport captured CO₂, in gas, solid or liquid form, for supplying the biological culture unit. The transportation of the captured CO₂ will depend on available infrastructure, ground and shipping transport costs, and so on.

It should be noted that feeding CO₂ directly to biological cultures may present various difficulties including the high cost to recover CO₂ from the absorption solution; high cost to transport the CO₂ to the biological culture site that is usually too large to be at the site of the plant generating the flue gas; problems with out-gassing of CO₂ in open systems; the fact that CO₂ cannot wait to be treated and many biological cultures stop at night; as well as efficiency, reliability and controllability issues.

In some embodiments of the present invention, the use of a bicarbonate solution for nutrient supply to a biological culture provides various improvements to these problems, such as lower cost for CO₂ recovery that is absorbed by the production of valuable products by the biological culture; facilitated transport and lower transport cost for bicarbonate solution or precipitate than for compressed CO₂; no out-gassing; ability of bicarbonate to be stocked during the night for improved process flexibility; and enhanced efficiency, reliability and controllability of the process.

It should be understood that embodiments of the present invention include treatment of CO₂ containing gas from any source, use of carbonic anhydrase in any form, use of any solvent or absorption compound that would not kill the biological culture, and the captured CO₂ may be transported to the biological culture in liquid, solid or slurry form.

The bio-products that are produced will depend on the biological culture and may include biofuels such as bio-diesel, bio-ethanol, other bio-alcohols, bio-oils for use as lubricants or nutritional supplements, pigments, vitamins, proteins, carbohydrates, as well as high value specialty chemicals that can be used as end-products and/or building blocks for the pharmaceutical, adhesives, plastics, or coatings industries, etc, that can be separated out of the culture. It should also be noted that, in some optional aspect, the bio-products could also be minimally- or non-processed biomass from the biological culture.

### EXAMPLES

### Example 1

In a first scenario, a typical 750 MW coal fired power plant was considered. This plant produces 4 million tons of CO₂ annually. The flue gas is treated to remove SOx and other contaminants, and sent to an absorber unit. This absorber captures CO₂ from the flue gas using sodium carbonate as an absorption solution and carbonic anhydrase as a bio-catalyst. Using an absorption solution and a biocatalyst, it is possible to capture up to 90% of CO₂ present in the flue gas. Considering the relatively low solubility of CO₂ in water, it would be nearly impossible to achieve required rate of absorption in pure water and absorption is sufficiently enhanced with an absorption solution and biocatalyst. The biocatalyst, in this case an enzyme, is an advantageous component because it greatly increases the absorption rate of the carbonate solution. Other solutions, like MEA or ammonia, are known to absorb CO₂ very fast, without the help of enzymes. However, such absorption solutions are not suitable for embodiments of the present invention because they form carbamate complexes reducing bicarbonate content in the absorption solution and thus diminishing the ion concentration available for the downstream microorganisms. Moreover, those solutions would impair algae or other biological culture growth and would have higher environment hazards in such applications. Once the absorption solution is transformed from carbonate to bicarbonate, it is sent in this example to an algae pond. Assuming that one mole of carbonate will capture one mole of CO₂ and generate 2 moles of bicarbonate, assuming a 0.5M Na₂CO₃ initial absorption solution, and assuming a 90% capture efficiency, 450,000 m³/day of absorption solution would be required to treat the 11,000 tons of CO₂ produced daily. Those 450,000 m³ of absorbed CO₂ solution, or at least a portion thereof, may be treated when flowing through the pond and then returned to the absorber. The bicarbonate solution will not rapidly degas as would CO₂ gas dissolved in simple aqueous solution, so we can assume that about 100% of the captured CO₂ will be available for algae growth. The solution may be handled in such a way as to prevent or reduce degassing. A pond of typical microalgae (Spirulina platensis as an example) will have a growth rate of 30 g/m²·day (dry weight). Knowing that about 1.8 g of CO₂ is used to generate 1 g of algae, 54 g of CO₂/m²·day will be used. To cope with the 10 ktons/day of captured CO₂ (11 ktons at 90% capture efficiency), a pond of about 13.6×13.6 km would be adequate. Assuming a 30 cm depth, this pound would have a volume of 54,000,000 m³. Multiple ponds may be used to provide the overall culture volume. About 30% of this pond will be harvested daily. The algae will be dried and the liquid fraction will return to the pond and to the absorber (450,000 m³/day). About 5,600 tons of dried algae will be obtained per day. Dried algae have an energy content of about 20 kJ/g, akin to lignite. At least a portion of this may be burnt in the power plant as an energy source. Ashes from the power plant and sewer sludge may also be used as fertilizer for the algae growth.

### Example 2

In this scenario, a small CO₂ emitting plant is considered. This plant produces 219 tons of CO₂ annually. The flue gas is treated to remove SOx and other contaminants, and sent to an absorber unit. This absorber captures CO₂ from the flue gas using sodium carbonate as an absorption solution and carbonic anhydrase as a bio-catalyst. Using an absorption solution and a biocatalyst, it is possible to capture up to 90% of CO₂ present in the flue gas. Once the absorption solution is transformed from carbonate to bicarbonate, it is sent in this example to an algae pond. Assuming that one mole of carbonate will capture one mole of CO₂ and generate 2 moles of bicarbonate, assuming a 0.5M Na₂CO₃ initial absorption solution, and assuming a 90% capture efficiency, 24.5 m³/day of absorption solution would be required to treat the 600 kg of CO₂ produced daily. Those 24.5 m³ of absorbed CO₂ solution, or at least a portion thereof, may be treated when flowing through the pond and then returned to the absorber. The bicarbonate solution will not rapidly degas as would CO₂ gas dissolved in simple aqueous solution, so we can assume that about 100% of the captured CO₂ will be available for algae growth. The solution may be handled in such a way as to prevent or reduce degassing. A pond of typical microalgae (Spirulina platensis as an example) will have a growth rate of 30 g/m²·day (dry weight). Knowing that about 1.8 g of CO₂ is used to generate 1 g of algae, 54 g of CO₂/m²·day will be used. To cope with the 540 kg/day of captured CO₂ (600 kg at 90% capture efficiency), a pond of about 10,000 m² (1 ha) would be adequate. Assuming a 30 cm depth, this pound would have a volume of 3,000 m³. Multiple ponds may be used to provide the overall culture volume. About 30% of this pond will be harvested daily. The algae will be dried and the liquid fraction will return to the pond and to the absorber (24.5 m³/day). About 300 kg of dried algae will be obtained per day. Dried algae have an energy content of about 20 kJ/g, akin to lignite. At least a portion of this may be burnt in the power plant as an energy source. Ashes from the power plant and sewer sludge may also be used as fertilizer for the algae growth.

### Example 3

Example 3 is similar to Example 1, but instead of having a large pond, vertical cylindrical photo bioreactors are used. Reactors having a production rate of 2,700 g of algae/m²·day would require a farm of 1.9 km×1.9 km to treat the flue gas. In a conventional system, the flue gas is directly bubbled throughout the algae culture. This causes the gas to experience a large pressure drop so a substantial amount of energy would be required to flow the gas through the bioreactors. Moreover, in that kind of system, about 50% of the CO₂ would be absorbed and the remaining would be directly emitted and lost to the atmosphere. In the case that a packed column absorber is used, as described here-above, the gas would pass throughout the absorber with a minimal pressure drop (and lower energy) and excellent capture efficiency (around 90%). A packed column provides a higher gas-liquid contact area than a bubbling photo bioreactor, thus enabling a higher CO₂ absorption efficiency.

In this system, the gas is not directly in contact with the algae culture, this prevents possible contamination of the culture by some eventual toxic gas contaminants. The bicarbonate enriched solution can then be pumped and channeled directly into the photo bioreactors. Bicarbonate concentration in this last setup will be much higher, thus more bicarbonate ions would be available to the algae culture. This should enable a higher algae growth rate and cell density. As a result it can reduce the farm footprint required for the installation. As for the pond system, a fraction of the algae culture is harvested. The solid phase (algae) and the liquid phase (bicarbonate depleted solution) are separated. Part of the liquid phase is returned to the absorber and the rest is returned to the photo bioreactors. The algae can then be dried and used as fuel or it can be processed to extract oil or other bio-product compounds. For example, algae like Phaeodactylum tricornutum contains about 30% oil (weight/dry weight).

Finally, the following references are incorporated herein by reference:
1. http://ion.chem.usu.edu/˜sbialkow/Classes/3600/Overheads/Carbonate/CO2.html. [En ligne] [Citation: 30 Sep. 2011.] http://ion.chem.usu.edu/˜sbialkow/Classes/3600/Overheads/Carbonate/CO2.html.
2. Chi, Z., O'Fallon, J. V. et Chen, S. Bicarbonate produced from carbon capture for algae culture. Trends in Biotechnology. 18 Jul. 2011.
3. Lide, David, R. Handbook of chemistry and physics 80th edition. s.l.: CRC Press, 1999-2000.
4. Jayasankar, R. et Valsala, K. K. Influence of different concentrations of sodium bicarbonate on growth rate and chlorophyll content of Chlorella salina. Journal of the marine biological association of India. 2008, Vol. 50, 1, pp. 74-78.
5. Sültemeyer, D. Carbonic anhydrase in eukariotic algae: characterization, regulation, and possible function during photosynthesis. Can. J. Bot. 1998, Vol. 76, pp. 962-972.
6. Azoz, Y. Effect of pH on inorganic carbon uptake in algal cultures. Applied and environmental microbiology. 1982, Vol. 43, 6, pp. 1300-1306.
7. Pimolrat, P., et al., et al. The effect of sodium bicarbonate concentrations on growth and biochemical composition of Chaetoceros gracilis Scutt. Katetsart university fisheries research bulletin. 2010, Vol. 34, 2.
8. Fradette, s. Ruel, J. Process and a plant for recycling carbon dioxide emissions from power plants into useful carbonated species. U.S. Pat. No. 7,596,952 B2 United States, Jun. 6 Oct. 2009.
9. Hazlebeck, D. A. et Dunlop, E. H. Photosynthetic oil production with high carbon dioxide utilization. U.S. Pat. No. 7,662,616 B2 United States, 16 Feb. 2010.
10. Chi, Z., et al., et al. Integrated system for production of biofuel feedstock. United State, 3 Feb. 2011.
11. Hu, Q. et Sommerfeld, M. Novel Chlorella species and uses therefor. US 20100021968 A1 United States, 28 Jan. 2010.
12. Sears, J. T. Method, apparatus and system for biodiesel production from algae. US 20070048848 A1 Unites States, 1 Mar. 2007.
13. Rasmussen, M. A., McNeff, C. V. et McNeff, L. C. Algae cultivation systems and methods. US20090126265 A1 United State, 21 May 2009.
14. Chi, Z., et al., et al. Integrated system for production of biofuel feedstock. WO 2010014797 A2 International, 4 Feb. 2010.
15. Wright, A. B., Lackner, K. S. et Ginster, U. Method and apparatus for extracting carbon dioxide from air. US 20110033357 A1 United States, 10 Feb. 2011.
16. Chinnasamy, S., et al., et al. Biomass Production Potential of a Wastewater Alga Chlorella vulgaris ARC 1 under Elevated Levels of CO2 and Temperature. Int. J. Mol. Sci. 2009, Vol. 10, pp. 518-532.
17. Futasugi, M et Gushi, K. Modified enzyme. EP 0 506 431 (A1) Europe, 1992.
18. Lewis, N. S. et Nocera, D. G. POwering the planet: chemical challenges in solar energy utilization. PNAS. 24 Oct. 2006, Vol. 103, 43, pp. 15729-15735.
19. Küppers, U. et Kremer, B. Longitudinal Profiles of Carbon Dioxide Fixation Capacities in Marine Macroalgae. Plant Physiol. 1978, Vol. 62, pp. 49-53.
20. James, S. C. et Boriah, V. Modeling Algae Growth in an Open-Channel Raceway. JOURNAL OF COMPUTATIONAL BIOLOGY. 2010, Vol. 17, 7, pp. 895-906.

In addition, various patents and applications are also incorporated herein by reference: U.S. Pat. No. 7,740,689, international PCT patent application Nos. PCT/CA2010/001212, PCT/CA2010/001213 and PCT/CA2010/001214, U.S. Pat. No. 6,908,507, U.S. Pat. No. 7,176,017, U.S. Pat. No. 6,524,843, U.S. Pat. No. 6,475,382, U.S. Pat. No. 6,946,288, U.S. Pat. No. 7,596,952, U.S. Pat. No. 7,514,056, U.S. Pat. No. 7,521,217, U.S. Patent Application No. 61/272,792, U.S. Patent Application No. 61/344,869, U.S. Patent Application No. 61/439,100, which are all currently held by the Applicant. All other patents and application held by the Applicant are also incorporated herein by reference. Various reactors and processes described in the preceding references may be used in connection with various processes and methods described in the present specification.

1-74. (canceled)

75. A method for dual biocatalytic conversion of CO₂ in a CO₂ containing gas into carbon containing bio-products by enzymatically catalyzing the hydration reaction of dissolved CO₂ into bicarbonate and hydrogen ions in the presence of carbonic anhydrase and metabolically converting the bicarbonate ions into the carbon containing bio-products in a biological culture.

76. The method of claim 75, comprising maintaining the dual biocatalytic conversion relatively constant and controlling a feeding of the bicarbonate ions to the biological culture in accordance with demands of the biological culture by retaining over-production of bicarbonate ions and feeding part of the over-production to the biological culture in accordance with nutrient demands of the biological culture.

77. The method of claim 76, wherein the over-production of the bicarbonate ions is retained in the form of carbonate precipitates.

78. A process for treating a CO₂ containing gas to produce carbon containing bio-products, comprising:
a) contacting an aqueous absorption solution comprising water and an absorption compound with the CO₂ containing gas in the presence of carbonic anhydrase;
b) enzymatically catalyzing the hydration reaction of dissolved CO₂ into bicarbonate and hydrogen ions within the aqueous absorption solution to produce a bicarbonate loaded solution;
c) metabolically converting the bicarbonate ions in the bicarbonate loaded solution into carbon containing compounds by a biological culture; and
harvesting and treating the biological culture to extract the carbon containing compounds and transforming the same into the carbon containing bio-products.

79. The process of claim 78, comprising controlling the temperature of the CO₂ containing gas before the step a) of contacting the aqueous absorption solution.

80. The process of claim 79, comprising cooling the CO₂ containing gas before the step a) of contacting the aqueous absorption solution.

81. The process of claim 80, comprising adjusting the pH of the aqueous absorption solution.

82. The process of claim 81, comprising removing contaminants from the CO₂ containing gas before the step a) of contacting the aqueous absorption solution.

83. The process of claim 82, comprising removing carbonic anhydrase from the bicarbonate loaded solution before the step c) of metabolically converting the bicarbonate ions.

84. The process of claim 83, comprising recycling a portion of the bicarbonate loaded solution to make up the aqueous absorption solution before the step a) of contacting.

85. The process of claim 78, comprising pre-treating the bicarbonate loaded solution before the step c) of metabolically converting the bicarbonate ions, to alter a solubility of the bicarbonate ions in the bicarbonate loaded solution to enhance precipitation thereof into carbonate precipitates.

86. The process of claim 85, wherein the pre-treating comprises altering the pH of the bicarbonate loaded solution and/or altering the temperature of the bicarbonate loaded solution.

87. The process of claim 85, wherein the pre-treating comprises adding a cationic co-precipitating agent.

88. The process of claim 85, comprising separating at least a portion of the precipitates, referred to as a precipitated solid fraction, from the bicarbonate loaded solution for downstream applications.

89. The process of claim 88, comprising adjusting an amount of the precipitated solid fraction to be redistributed to the biological culture in accordance with monitoring growth cycles of the biological culture.

90. The process of claim 89, comprising mixing the amount of the precipitated solid fraction to be redistributed with a liquid containing nutrients for the biological culture to form a supplemental bicarbonate nutrient stream for supply to the biological culture.

91. The process of claim 90, wherein the liquid is derived from a wastewater source.

92. The process of claim 85, comprising desorbing CO₂ from at least a portion of the bicarbonate loaded solution and/or of the carbonate precipitates to generate a pure CO₂ gas stream and an ion-depleted solution recyclable as a portion of the aqueous absorption solution.

93. The process of claim 90, wherein the step d) of harvesting and treating the biological culture also produces a separated solution, the process comprising recycling a portion of the separated solution as the liquid containing nutrients to form the supplemental bicarbonate nutrient stream.

94. The process of claim 78, comprising recycling a remaining portion of the separated solution to make up the aqueous absorption solution before the step a) of contacting.

95. The process of claim 78, wherein the step c) of metabolically converting the bicarbonate ions also produces a bicarbonate-depleted solution, the process comprising recycling at least a portion of the bicarbonate-depleted solution to make up the aqueous absorption solution before the step a) of contacting.

96. The process of claim 78, wherein the step d) comprises transforming the carbon containing compounds into bio-oils for lubrication, liquid fuels for energy supply, or a combination thereof.

97. The process of claim 78, wherein the step d) also comprises extracting biomass for use as solid fuel and/or feedstock.

98. The process of claim 78, wherein the step d) also comprises extracting a nutrient fraction to be supplied to the biological culture.

99. An apparatus for dual biocatalytic conversion of CO₂ gas in flue gas into carbon containing bio-products, comprising:
a) an enzymatic bicarbonate production and CO₂ gas absorption unit comprising:
(i) a gas inlet for receiving the flue gas;
(ii) a liquid inlet for receiving an aqueous absorption solution;
(iii) a reaction chamber configured for receiving the flue gas and the aqueous absorption solution such for contact in the presence of carbonic anhydrase for catalyzing the hydration reaction of dissolved CO₂ from the flue gas into bicarbonate and hydrogen ions to produce a bicarbonate loaded solution and a treated gas;
(iv) a gas outlet for releasing the treated gas; and
(v) a liquid outlet for releasing the bicarbonate loaded solution;
b) a biological culture unit comprising:
(i) an inlet for receiving the bicarbonate loaded solution;
(ii) a culture compartment for metabolically converting the bicarbonate ions by a biological culture into carbon containing bio-products; and
(iii) an outlet for releasing biological culture material containing the carbon containing bio-products from the culture compartment; and
(iv) an extraction unit for extracting at least some of the carbon containing bio-products from the released biological culture material.

100. A dual biocatalytic formulation for conversion of CO₂ in a CO₂ containing gas into carbon containing bio-products, comprising water; CO₂ dissolved in the water; carbonic anhydrase in suspension in the water in sufficient amount to catalyze the hydration reaction of dissolved CO₂ into bicarbonate and hydrogen ions in the water in a nutritive bicarbonate concentration; and biological culture material in the water in sufficient amount to have sustained metabolic activity in the nutritive bicarbonate concentration for conversion of the bicarbonate ions into the carbon containing bio-products.

101. The process of claim 78, wherein the biological culture produces at least part of the enzyme for use in the enzymatic CO₂ capture.

102. The process of claim 78, wherein the biological culture comprises a micro-organism culture, green algae, an alkaliphilic micro-organism culture, a halophilic micro-organism culture, an euglena culture, purple sulfur and non-sulfur bacteria culture, green sulfur and non-sulfur bacteria culture, nitrosomonas bacteria culture, nitrobacter bacteria culture, and/or methanogen archaea culture, strains thereof, variants thereof or mixtures thereof.

103. The process of claim 102, wherein the micro-organism culture is cyanobacteria.

104. The process of claim 103, wherein the cyanobacteria are Phormidium ambiguum, Phormirium orientalis, Microcoleus sp or a combination thereof.

105. The process of claim 78, wherein the carbonic anhydrase is immobilized or entrapped on or in packing or internals of a reactor.

106. The process of claim 78, wherein the carbonic anhydrase is associated with free floating particles flowing through a reactor, the carbonic anhydrase being immobilized, bonded, entrapped and/or coated onto the particles using a stabilization material.

107. The process of claim 78, wherein the carbonic anhydrase is present as aggregates or crystals in suspension in an aqueous liquid.
